(19) United States
(12) Patent Application Publication
(10) Pub. No.: US 2025/0166827 A1
(43) Pub. Date: May 22, 2025

(54) MULTIMODAL FOUNDATION MODEL FOR PATIENT RISK STRATIFICATION

(71) Applicant: GE Precision Healthcare LLC, Waukesha, WI (US)

(72) Inventors: Soumya Ghose, Niskayuna, NY (US); Sanghee Cho, Schenectady, NY (US); Fiona Mary Ginty, Saratoga Springs, NY (US); Sriram Boppana, Redmond, WA (US); SM Shajedul Hasan, Rexford, NY (US); Erhan Bas, Sammamish, WA (US); Thiruvarul Selvan Senthivel, Snoqualmie, WA (US)

(21) Appl. No.: 18/949,548

(22) Filed: Nov. 15, 2024

Related U.S. Application Data
(60) Provisional application No. 63/602,004, filed on Nov. 22, 2023.

(51) Int. Cl. G16H 50/20; G16H 10/60; G16H 50/30

(52) U.S. Cl. CPC G16H 50/20; G16H 10/60; G16H 50/30

(57) ABSTRACT

The described technology is generally directed towards automated and dynamic creation of a recommended treatment for a patient's medical condition. A potential treatment can be generated by application of patient data to a multimodal model representing radiomic data and pathomic data integrated with transcriptomic data. The potential treatment can be applied to a probability model to determine a risk score for the potential treatment. The probability model comprises a collection of treatments and medical conditions, in conjunction with respective measures of success of application of a respective treatment to a respective condition. The multimodal model can comprise a network of nodes and edges, wherein the nodes represent knowledge of a patient condition, medical knowledge/medical research regarding the respective patient conditions, and the like. An output of the multimodal model is one or more recommended treatments for the medical condition.

### CROSS-REFERENCE TO RELATED APPLICATIONS

This application claims priority to U.S. Provisional Patent Application No. 63/602,004 filed on Nov. 22, 2023, entitled “MULTIMODAL FOUNDATION MODEL FOR PATIENT RISK STRATIFICATION”. The entireties of the aforementioned application are incorporated by reference herein.

### TECHNICAL FIELD

This application relates to systems and techniques facilitating analysis and presentation of medical information and treatment recommendations regarding a patient.

### BACKGROUND

Technological advancements in the last years have prompted the medical world to strive for precision healthcare. The goal of precision healthcare comprises the ability for a caregiver to retrieve precise information (about the patient and/or about treatment) at the right time and place. One way in which this ability is needed relates to the time available to review a patient's medical data tends to become shorter as the workload and working requirements of medical staff increases (e.g., as radiologists and clinicians work intensifies). Another way is the need for ready and easy querying sources of information, with accurate/well considered treatment recommendations.

The above-described background is merely intended to provide a contextual overview of some current issues and is not intended to be exhaustive. Other contextual information may become further apparent upon review of the following detailed description.

### SUMMARY

The following presents a simplified summary of the disclosed subject matter to provide a basic understanding of one or more of the various embodiments described herein. This summary is not an extensive overview of the various embodiments. It is intended neither to identify key or critical elements of the various embodiments nor to delineate the scope of the various embodiments. The sole purpose of the Summary is to present some concepts of the disclosure in a streamlined form as a prelude to the more detailed description that is presented later.

In one or more embodiments described herein, systems, devices, computer-implemented methods, configurations, apparatus, and/or computer program products are presented to implement a multimodal model in combination with a probability model to diagnose a medical condition of a patient and further determine a recommended treatment for the medical condition.

According to one or more embodiments, a system is presented, wherein the system comprises at least one processor, and at least one memory coupled to the at least one processor and having instructions stored thereon, wherein the system can be configured to identify a patient medical condition and further identify a recommended treatment for the medical condition. In response to the at least one processor executing the instructions, the instructions facilitate performance of operations, comprising: receiving pathomic information, radiomic information, and transcriptomic information, further generating a multimodal model, wherein the multimodal model can be generated based on combining the received pathomic information, radiomic information, and transcriptomic information, and further applying patient data to the multimodal model, wherein the patient data pertains to a medical condition of a patient. In a further embodiment, the operations can further comprise generating, based on application of the patient data to the multimodal model, a recommended treatment, wherein the recommended treatment can be based on at least one node, in a network of nodes included in the multimodal model, having sufficient similarity to the patient data.

In another embodiment, the operations can further comprise determining a risk score for the recommended treatment, wherein the risk score presents a measure of a successful treatment outcome for the recommended treatment regarding the patient's medical condition, wherein the risk score can be determined based on applying the recommended treatment to a probability model, and further presenting the recommended treatment and risk score for review.

In another embodiment, the operations can further comprise receiving a confirmation to implement the recommended treatment; and further updating the patient data in accordance with the recommended treatment being applied to treat the patient's medical condition.

In a further embodiment, the operations can further comprise utilizing natural language programming to facilitate interaction with the presented recommended treatment and risk score.

In another embodiment, wherein the interaction can be via at least one of a mouse and cursor, interactive display, or speech-based interaction.

In a further embodiment, the multimodal model can comprise at least one of a visual language model, a large language model, a Bayesian network, a convolutional neural network, graph neural network, or a knowledge graph.

In another embodiment, the multimodal model can comprise the network of nodes and edges connected to a set of potential treatments, a respective node in the network of nodes represents content pertaining to the patient's medical condition or medical knowledge regarding a medical condition, wherein the medical knowledge pertains or does not pertain to the patient's medical condition.

In a further embodiment, the probability model can comprise at least one of a visual language model, a large language model, a Bayesian network, a convolutional neural network, graph neural network, or a knowledge graph.

In another embodiment, wherein the recommended treatment is a first recommended treatment and the risk score is a first risk score, wherein the operations further comprise generating, based on application of the patient data to the multimodal model, a second recommended treatment, wherein the second recommended treatment is based on at least one node in the sequence of nodes having sufficient similarity to the patient data, and further determining a second risk score for the second recommended treatment, wherein the second risk score presents a measure of a successful treatment outcome for the second recommended treatment regarding the patient's medical condition, wherein the second risk score is determined based on applying the second recommended treatment to a probability model. In a further embodiment, the operations can further comprise ranking the first recommended treatment and the second recommended treatment based on the first risk score and the second risk score, and further presenting the ranking of the first recommended treatment and first risk score, and the second recommended treatment and the second risk score.

In another embodiment, the multimodal model can comprise transcriptomic data pertaining to the medical condition of the patient in combination with at least one of radiology data pertaining to the medical condition of the patient, or pathology data pertaining to the medical condition of the patient.

In a further embodiment, the transcriptomic data pertaining to the medical condition of the patient can further comprise at least one of proteomic information, single-cell RNA sequencing field (scRNAseq) information, an autofluorescence image, matrix-assisted laser desorption/ionization (MALDI) information, spatial transcriptomic information, multiplexed error-robust fluorescence in situ hybridization (MERFISH), spatial gene expression, or metaboliomic information.

In further embodiments, a computer-implemented method is provided, wherein the method comprises generating, by a device comprising at least one processor, a multimodal model, wherein the multimodal model can be generated based on at least one of a medical condition of a patient, historical data pertaining to the medical condition, imaging data pertaining to the medical condition, or medical knowledge pertaining to the medical condition, and in a further embodiment, the method further comprising determining, by the device, a potential treatment to address the medical condition of the patient, wherein the treatment is an output of the multimodal model.

In a further embodiment, the computer-implemented method can further comprise determining, by the device, a probability of the potential treatment successfully addressing the medical condition of the patient, wherein the probability is determined based on application of the potential treatment to a probability model, wherein the probability model comprises a collection of treatments in conjunction with success of application of a respective treatment in the collection of treatments to a respective medical condition, wherein the probability is assigned to a risk score for the potential treatment of the patient's medical condition, and further presenting, by the device, a recommendation for treatment of the patient's medical condition, wherein the recommendation comprises the potential treatment in conjunction with the risk score.

In an embodiment, wherein the multimodal model can be one of a visual language model, a large language model, graph neural network, or a Bayesian network.

In an embodiment, the visual language model can comprise transcriptomic data pertaining to the medical condition of the patient in combination with at least one of radiology data pertaining to the medical condition of the patient, or pathology data pertaining to the medical condition of the patient.

In an embodiment, the multimodal model and the probability model can be combined to form a prediction model, the prediction model comprises at least one of a visual language model, a large language model, graph neural network, or a Bayesian network.

In another embodiment, the risk score indicates a risk stratification of at least one of the medical condition of the patient or the recommended treatment.

Further embodiments can include a computer program product stored on a non-transitory computer-readable medium and comprising machine-executable instructions, wherein in response to being executed, the machine-executable instructions cause a system to perform operations, comprising generating a multimodal model, wherein the multimodal model is generated based on at least one of a medical condition of a patient, historical data pertaining to the medical condition, imaging data pertaining to the medical condition, or medical knowledge pertaining to the medical condition, and further determining a potential treatment to address the medical condition of the patient, wherein the treatment is an output of the multimodal model.

In an embodiment, the operations can further comprise determining a probability of the potential treatment successfully addressing the medical condition of the patient, wherein the probability is determined based on application of the potential treatment to a probability model, wherein the probability model comprises a collection of treatments in conjunction with success of application of a respective treatment in the collection of treatments to a respective medical condition, wherein the probability is assigned to a risk score for the potential treatment of the patient's medical condition, and further presenting a recommendation for treatment of the patient's medical condition, wherein the recommendation comprises the potential treatment in conjunction with the risk score.

In a further embodiment, the multimodal model and the probability model combine to form a prediction model, the prediction model comprises at least one of a visual language model, a large language model, graph neural network, or a Bayesian network.

### DESCRIPTION OF THE DRAWINGS

One or more embodiments are described below in the Detailed Description section with reference to the following drawings.

FIG. 1 illustrates a system that can be utilized to implement a multimodal model in conjunction with probability analysis to determine a patient treatment, according to at least one embodiment.

FIG. 2 presents a schematic of an example process for generating one or more recommended treatments for a patient, in accordance with one or more embodiments.

FIG. 3 illustrates implementation of a probability model to further implement a recommendation, in accordance with an embodiment.

FIG. 4 illustrates a computer-implemented method for generating a recommended treatment for a patient's condition, in accordance with an embodiment.

FIG. 5 illustrates a computer-implemented method for implementing a recommended treatment for a patient's condition, in accordance with an embodiment.

FIG. 6 illustrates a computer-implemented method for generating a VLM incorporating radiomics, pathomics, transcriptomics, and suchlike, in accordance with an embodiment.

FIG. 7 presents an example computer-implemented method for implementing a multimodal model in conjunction with a probability model to identify a medical condition and further identify/recommend a treatment, in accordance with an embodiment.

FIG. 8 presents an example computer-implemented method for utilizing a multimodal model in conjunction with a probability model to identify a medical condition and further identify/recommend a treatment, in accordance with an embodiment.

FIG. 9 presents an example computer-implemented method for utilizing a multimodal model in conjunction with a probability model to identify a medical condition and further identify/recommend a treatment, in accordance with an embodiment.

FIG. 10 is a block diagram illustrating an example computing environment in which the various embodiments described herein can be implemented.

FIG. 11 is a block diagram illustrating an example computing environment with which the disclosed subject matter can interact, in accordance with an embodiment.

### DETAILED DESCRIPTION

The following detailed description is merely illustrative and is not intended to limit embodiments and/or application or uses of embodiments. Furthermore, there is no intention to be bound by any expressed and/or implied information presented in any of the preceding Background section, the Abstract, and/or in the Detailed Description section.

One or more embodiments are now described with reference to the drawings, wherein like reference numerals are used to refer to like elements throughout. In the following description, for purposes of explanation, numerous specific details are set forth in order to provide a thorough understanding of the various embodiments. It is to be appreciated, however, that the various embodiments can be practiced without these specific details, e.g., without applying to any particular networked environment or standard. In other instances, well-known structures and devices are shown in block diagram form in order to facilitate describing the embodiments in additional detail.

It is to be understood that when an element is referred to as being “coupled” to another element, it can describe one or more different types of coupling including, but not limited to, chemical coupling, communicative coupling, electrical coupling, electromagnetic coupling, operative coupling, optical coupling, physical coupling, thermal coupling, and/or another type of coupling. Likewise, it is to be understood that when an element is referred to as being “connected” to another element, it can describe one or more different types of connecting including, but not limited to, electrical connecting, electromagnetic connecting, operative connecting, optical connecting, physical connecting, thermal connecting, and/or another type of connecting.

As used herein, “data” can comprise metadata. Further, ranges A-n are utilized herein to indicate a respective plurality of devices, components, signals etc., where n is any positive integer.

### Overview

Various technologies and techniques are presented herein regarding the utilization of a series/collection/multiple of visual language foundation models (VLM) for radiology, pathology and clinical data to build a multimodal predictive model.

In an embodiment, a multimodal model can comprise a multimodal foundational model comprising radiology, pathology, and clinical data/variable(s) and further extended to include transcriptomics data, including, in a non-limiting list, autofluorescence images, matrix-assisted laser desorption/ionization (MALDI), proteomics, single-cell RNA sequencing field (scRNAseq), spatial transcriptomics data (e.g., multiplexed error-robust fluorescence in situ hybridization (MERFISH), spatial gene expression, and suchlike) and the like. In an embodiment, by generating a multimodal model that includes transcriptomic information/data, analysis, diagnosis, and potential treatment of a patient's condition can expand upon the medical knowledge only available from radiology and pathology information. Transcriptomic data can provide knowledge at the molecular/DNA/RNA/micro-level, to supplement the macro-level data that is available with radiology (organ-level) and pathology (cellular-level) information. Accordingly, for example, diagnosis and treatment of a cancerous tumor can be based on RNA information (e.g., indicating an environmental response of the DNA), which can provide insight into the patient condition that is not available with macro-level based diagnosis. Further, insight into how a tumor may be evolving can also be gleaned from the organ level information in combination with the cellular level information and further in combination with the DNA level information.

The multimodal model can be utilized to generate a patient specific risk score. In an embodiment, the risk score can be generated from the available data (e.g., whether presented to caregiver or available as background information), wherein such data can include, in a non-limiting list, radiology data/variables, pathology data/variables, clinical data/variables, as well as data relating to any of protein, gene, tissue, and suchlike, which can be utilized to generate the patient risk score, as further described.

A multimodal model created per the one or more embodiments presented herein can have wide-ranging application in any of the healthcare and biopharmaceutical industries, for example, in a non-limiting list: risk stratification of patients for immunotherapy, theranostics, Alzheimer disease treatment, contrast development, vaccine development, drug development procedures, and suchlike.

Various technologies and techniques can be utilized to provide a probabilistic expectation of an outcome(s) regarding a patient care pathway per prior and current medical images, clinico-pathological variables, and suchlike, wherein the prior information can be historical data capturing previous interaction with the multimodal model/prior risk scores/assessments, prior created images, previously obtained measurements/imaging, prior determined scores, and suchlike.

In an embodiment, probabilistic expectation can be determined by utilizing a probability-based model (e.g., a decision tree, a random forest, a Bayesian network), knowledge graphs, a graph neural network, a graph convolutional neural network, and similar technologies/techniques. Probability techniques can be utilized to ascertain population statistics for a given patient care pathway (e.g., a particular edge/node combination in the multimodal model) based on current/prior knowledge.

In an embodiment, the respective risk score(s) generated from the probability model can be utilized to generate/rank one or more recommendations regarding treatment of a patient and their medical condition. Implementing the one or more probability techniques can provide an indication of applicability/success of the one or more clinical recommendations. In a further embodiment, the probability analysis can draw from any of, in a non-limiting list, medical imaging data, population statistics, patient priors, clinico-pathological variables, and suchlike. Probability technologies facilitate determination/analysis of any deviations in the one or more recommendations from standard/usual clinical practice based on population statistics, and suchlike. Accordingly, by applying probability statistics to the recommended treatment(s) determined from the multimodal model, the risk of erroneous treatment recommendation(s) can be reduced. For example, a first recommended treatment is derived by/from the multimodal model, but when compared to prior implementation of the first recommended technique, it was found to cause more complications than it resolved, hence, a low probability of success can be applied to the recommended technique. Alternatively, a second recommended treatment, might be presented with a 50% probability, indicating that while the level of success in implementing the second recommended treatment is not high/guaranteed, the level of probability might be sufficiently high enough for the caregiver/researcher to use the second recommended treatment as a foundation for further review (e.g., in pharmaceutical development). Hence, application of the combination of the recommended treatment(s) and risk assessment can be beneficial both with regard to reducing errors commonly present when utilizing visual language models in conjunction with large language models, as well as providing a basis for further study/analysis/research regarding a recommended treatment/outcome having a lower probability of success/degree of confidence that clinical application generally requires.

Implementation and integration of radiomics, pathomics, transcriptomics, clinical variables, clinical guidelines, next step treatment recommendation(s) in conjunction with a multimodal model and a probability tree/knowledge graph created from medical images, clinical variables, clinical guidelines, etc., enables a caregiver to traverse all possible outcomes for a patient based on multimodal image, data, clinical guidelines, etc., in a generative artificial intelligence (AI)/machine language (ML) framework based on population statistics, and suchlike.

In an embodiment, a clinical guidance system (CGS) can be utilized to communicate with clinicians and patients in a natural language environment. By utilizing natural language processing (NLP) techniques, the clinical guidance system can be configured to present clinically explainable disease state along with any recommended next course of action(s) based on/in conjunction with pertinent medical images, clinico-pathological variables, population statistics, clinical guidelines, and suchlike.

Utilizing a natural language-based interaction can, in a non-limiting list:
(a) reduce the time a caregiver interacts with the CGS;
(b) by employing consistent/comparable techniques across multiple application of the CGS, variability in a clinical decision-making process can be reduced/mitigated;
(c) a checklist can be implemented for the clinician based on prior population statistics that is tractable and can enable improved reimbursement; and
(d) present patient-related information to the patient enabling the patient to gain better understanding of their condition/treatment(s), improved/educated decisions regarding giving consent to undergo the recommended clinical procedure, build patient confidence in their treatment/medical team as a function of the transparency of information imparted by the CGS, and suchlike.

An advantage of the one or more systems, computer-implemented methods and/or computer program products presented herein can be generating a risk scored recommended treatment enabling a caregiver to expeditiously assess a patient's condition and proposed treatment, while minimizing the amount of time the medic has to engage with the data to make the diagnosis, decision, etc. Further, implementing natural language processing for interaction with the system, enables an improved interaction with the system by a caregiver/patient.

It is to be appreciated that while the various embodiments presented herein are directed towards application of AI/ML to generate one or more recommended treatments and NLP interaction, the embodiments are equally applicable to any comparable use, e.g., presentation of veterinary information, presentation of financial data, failure diagnostics, and suchlike.

### Treatment Recommendation System

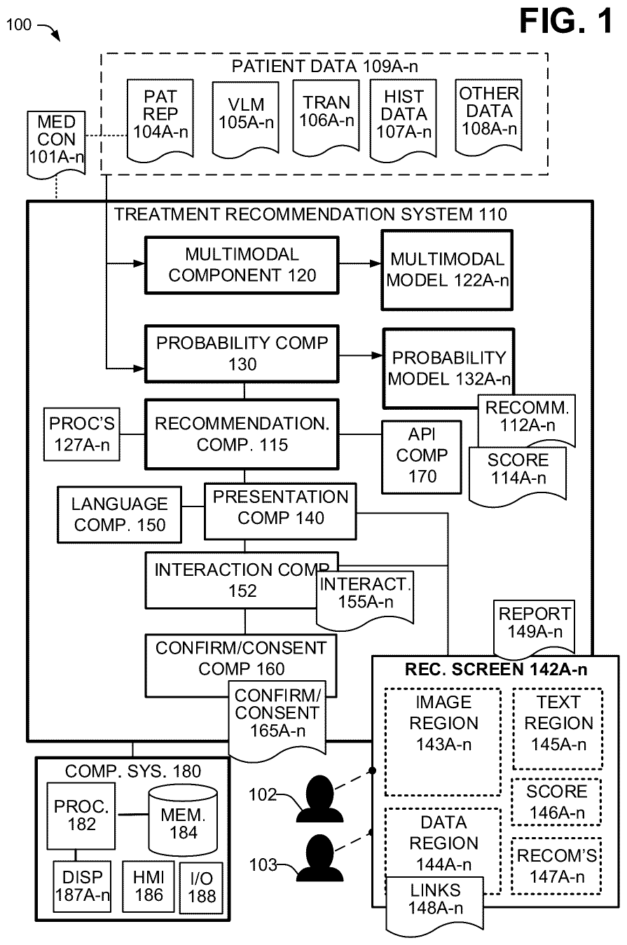

Turning now to the drawings, FIG. 1 illustrates a system 100 that can be utilized to utilize a multimodal model in conjunction with probability analysis to determine a patient treatment, according to at least one embodiment.

System 100 comprises a treatment recommendation system (TRS) 110 configured to:
(a) generate a multimodal model 122A-n comprising information from a plurality of sources, domains, etc.;
(b) multimodal model 122A-n enables a medical treatment for a patient 102 to be determined/recommended;
(c) probability analysis can be performed to assess applicability of the recommended medical treatment for the patient 102's medical condition 101A-n;
(d) in response to a determination that the recommended medical treatment is applicable, the medical treatment can be recommended and presented via a screen 142A-n, wherein the recommendation and any pertinent information can be presented in a manner facilitating natural language interaction with TRS 110;
(e) interaction between a caregiver 103 and/or patient 102 can proceed to increase knowledge of the medical condition 101A-n and/or recommended treatment to facilitate provision of an educated informed consent to undergo the recommended treatment, if needed; and
(f) interaction of the patient 102/caregiver 103 with TRS 110 can be monitored to enable further training of the multimodal model 122A-n/probability model 132A-n.

The term medical condition 101A-n is utilized herein with regard to a known medical condition, an inferred medical condition (e.g., a best guess by caregiver 103), an unknown/previously undiagnosed medical condition, and the like. Medical condition 101A-n can be any physical condition, mental condition, and the like, pertaining or potentially pertaining to patient 102.

As shown in FIG. 1, a plethora of information can be received at TRS 110, including, in a non-limiting list, patient data/reports 104A-n (e.g., patient personal data such as name, age, etc.; medical data such as measurements, doctor notes, etc.; and/or medical images), visual language model 105A-n (e.g., comprising images, radiomics, pathomics, and suchlike), transcriptomics data 106A-n (e.g., including, in a non-limiting list, autofluorescence images, MALDI information, proteomic information, single-cell RNA sequencing field (scRNAseq) information, spatial transcriptomics data (e.g., MERFISH), spatial gene expression, and suchlike), historical data 107A-n, and any other data/information 108A-n pertinent to patient 102's condition 101A-n and derivation of a recommended treatment 112A-n.

Historical data 107A-n can include any prior interactions 155A-n, selected hyperlinks 148A-n, previously generated models 122A-n/132A-n, and suchlike, as well as medical studies, papers, etc., in patient information 109A-n, pertaining to patient 102′s condition 101A-n. Accordingly, processes 127A-n can be trained as a function of prior interactions with particular information by a caregiver 103A-n. Hence, when reviewing a first recommended treatment 112A, if further information was sought by the caregiver 103, the importance of the further information (e.g., reports similar to a patient report 104P are typically utilized by caregiver 103A) can be established such that after repeated/separate instances of interaction, a process 127F associated with report 104P can be trained to weight/bias report 104P when determining a treatment 112A-n. Further, by frequently training the respective processes 127A-n, as new information is received, e.g., research is obtained regarding a parameter/measure/criteria being determined by the medical community to be playing a greater/lesser role in diagnosing a condition 101A-n, the respective processes 127A-n can be trained to reflect the greater/lesser importance of a particular criteria, etc., in diagnosing a condition 101A-n, and whether information relating to the particular criteria should be included/removed during creation of information for presentment on screen(s) 142A-n.

Any of information/data 104A-n-108A-n can be received (e.g., via an I/O 188, as further described) and stored at the TRS 110 (e.g., in memory 184, as further described). For the sake of readability, the respective data, images, etc., comprising 104A-n-108A-n are grouped under the term patient data 109A-n. Patient data 109A-n can comprise text, alphanumerics, numbers, single words, phrases, short statements, long statements, expressions, context, tables, JavaScript Object Notation (JSON) objects, images, etc., rendering the respective content of the patient data 109A-n to be searchable. Information 104-108 can be of any suitable format/filetype, such as a PDF, DICOM PDF, word processor document, spreadsheet, and suchlike, and can further include medical condition data 101A-n regarding the patient 102, medical history, medication history, vital signs, lab exam results, radiology and pathological results, medical consult assessments, and suchlike, and further include any images associated with treatment of patient 102, e.g., magnetic resonance imaging (MRI), X-ray image, mammogram image, scans, and suchlike.

While not shown, patient reports 104A-n (and other data, images, metadata, and suchlike) can be generated by an electronic medical record (EMR) system, or any system configured to stream, store, or generate information, data, etc., regarding a patient 102's condition 101A-n.

A recommendation component 115 can be included in TRS 110, wherein, recommendation component 115 can be configured to generate one or more recommended treatments 112A-n. Recommendation component 115 can be configured to interact/interface with other components included in TRS 110 to facilitate execution of steps (a)-(f) presented above.

TRS 110 can include a multimodal component 120 configured to

generate/implement one or more multimodal models 122A-n pertinent to analyzing/assessing patient 102's condition 101A-n, and further generating one or more recommended treatments 112A-n for patient 102. Multimodal models 122A-n can comprise of visual language models (VLMs) representing radiomics/pathomics, and other pertinent modalities, integrated with transcriptomic data 106A-n. Radiomics generally relates to a quantitative analysis of medical images (e.g., patient 102's oncology images, MRI's, X-rays, etc.), via application of AI/ML/mathematical analysis. Pathomics generally relates to utilizing AI/ML to analyze images with regard to making a diagnosis/manage patient care, as pertains to the field of histopathology. Transcriptomics generally relates to the study of an entity's cell, tissue, etc., at the ribonucleic acid (RNA) molecule level, and as mentioned previously.

In an embodiment, early/middle fusion may be used between radiology, pathology, proteomics, metaboliomics, scRNAseq and spatial transcriptomics to improve data heterogeneity and quality across a multimodal model 122A-n. For example, scRNAseq and spatial transcriptomics may be combined using deep generative models to increase spatial and gene expression resolution to investigate differential gene expression and predict treatment response and determination of a recommended treatment 112A-n. Further, differential gene expression can be further used for drug selection and predicting treatment response of a recommended treatment 112A-n for a drug in combination with radiomic information/features (e.g., radiomics 210A-n, as further described) and pathomic information/features (e.g., pathomics 212A-n, as further described). Furthermore, differential gene expression, along with radiomics information and pathomics information can be used for drug, vaccine, and/or contrast agent development for diseases, e.g., in a non-limiting list of oncology, cardiology, and neurology.

In another embodiment, reinforcement learning can be used for fine tuning the multimodal model 122A-n. In a further embodiment, self-supervised models, self-distillation with no labels models, and the like (e.g., distillation with no labels (DINO)) can be used for automatic label generation and training the multimodal model 122A-n.

In another embodiment, the multimodal model 122A-n can be a visual language model, whereby feature images and corresponding description can be utilized with the multimodal model 122A-n, such that at one or more regions of the multimodal model 122A-n (e.g., at a node, at an edge, at a decision point, and the like) can be further presented with the feature images/description to enable caregiver 103/patient 102 to further understand why a particular treatment was recommended, a probability of success of the treatment (as further described), factors involved in a particular decision at a node, and the like. By utilizing multimodal model 122A-n as a visual language model, the caregiver 103/patient 102 can readily discern reasoning behind a recommended treatment 112A-n, rendering use of the multimodal model 122A-n to be easier to conduct than non-visual representation, and further may expedite the diagnosis/treatment process as the caregiver 103/patient 102 can readily discern the decisions/logic behind the diagnosis/treatment.

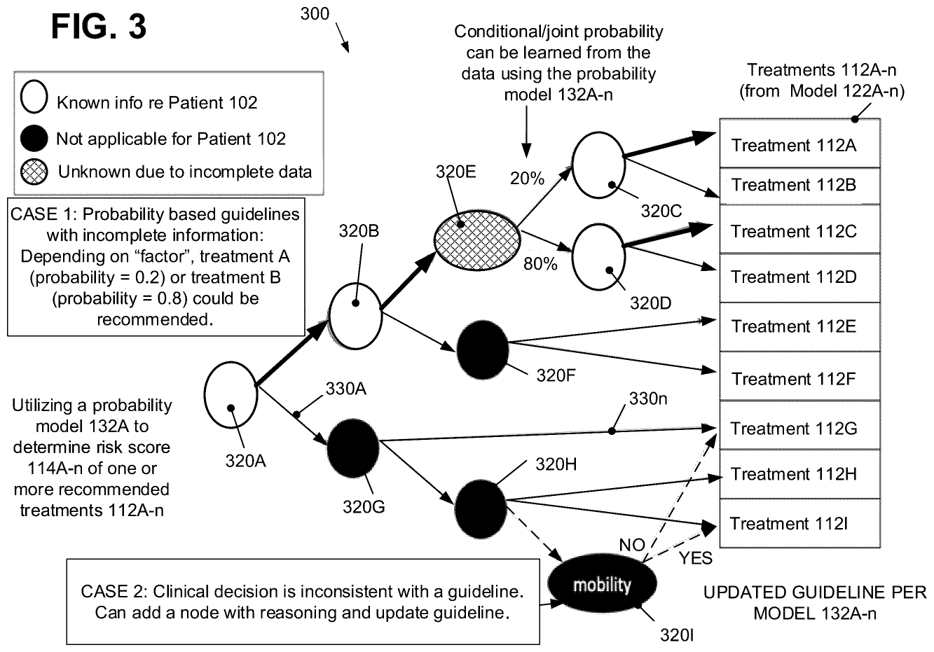

TRS 110 can further include a probability component 130 configured to apply available knowledge to the recommended treatment 112A-n to further determine whether the recommended treatment 112A-n is an acceptable/effective treatment for patient 102's condition 101A-n. In an embodiment, the probability component 130 can utilize a probability tree/model 132A-n, wherein the probability model 132A-n can be generated from the patient data 104A-n and historical data 107A-n (e.g., prior interactions with TRS 110, approved recommendations 112A-n, denied recommendations 112A-n, further data analysis performed by caregiver 103 to assess the quality of the recommended treatment 112A-n, and suchlike). With reference to FIG. 3, the probability model 132A-n can comprise of nodes 320A-n and edges 330A-n, wherein the edges 330A-n connect respective nodes 320A-n representing known/unknown information. As further described, the probability component 130 can be configured to generate the probability model 132A-n and further determine probability of a recommended treatment 112A-n being applicable/effective. In an aspect, the combination of the multimodal model 122A-n with the probability model 132A-n can be considered to form a prediction model, wherein the prediction model is generated based on application of the patient data 109A-n, and further, the prediction model generates one or more recommended treatments, wherein one or more recommended treatments are generated with a respective risk score enabling risk stratification of the one or more recommended treatments.

In an embodiment, the probability component 130 can review current/prior knowledge (e.g., in historical data 107A-n) regarding one or more aspects that pertain to patient 102's medical condition 101A-n, and hence determine a probability of a successful outcome for a given treatment. The treatment probabilities can be compared with the recommendations 112A-n generated by the multimodal component 120/multimodal model 122A-n to determine whether one or more recommendations 112A-n should be implemented. Hence, while the multimodal model 122A-n is configured to generate a recommendation 112A-n (e.g., via LLM process 127A-n), the applicability to patient 102's medical condition 101A-n is further assessed, e.g., based on prior knowledge in historical data 107A-n. A respective risk/suitability score 114A-n can be generated from the one more recommendations 112An, enabling the recommendations 112A-n to be ranked according to risk/suitability, e.g., a first recommendation 112F is determined to be 80% applicable to patient 102's condition 101A-n while a second recommendation 112D is determined to have 20% applicability, accordingly implementation of treatment 112F can be recommended by the recommendation component 115 over implementing treatment 112D.

TRS 110 can further include various processes 127A-n, wherein the processes 127A-n can include various AI and ML technologies and techniques available to be implemented by the one or more components included in TRS 110. For example, processes 127A-n can include large language model (LLM) technologies, probability trees, and suchlike. Further, during probability analysis of the recommended treatments, vector and similarity analysis can be performed to enable comparison between respective elements of the recommended treatment and prior treatments, etc., in historical data 107A-n, and suchlike.

As further shown, TRS 110 can include a presentation component 140 configured to control/configure presentation of the various patient data 109-n, recommended treatments 112A-n, scores 114A-n, and suchlike, on one or more recommendation screens 142A-n. As shown, in an example embodiment, a recommendation screen 142A-n can include a variety of regions such as an image region 143A-n configured to present various images pertaining to patient 102's medical condition 101A-n (e.g., X-rays, MRIs, etc.), a data region 144A-n configured to present various measurements, etc., a text region 145A-n to present information such as a summary of patient's condition 101A-n along with hyperlinks to further data if required, a score region 146A-n, and a recommendation region 147A-n. In an embodiment, respective summaries of information, data, etc., may be presented, with hyperlinks/links 148A-n enabling connection to further information having greater detail for review. With a multimodal model comprising a visual language model, the visual language model can be configured to provide images (e.g., images in image region 143A-n) in conjunction with one or more descriptions (e.g., short text statements explaining features provided in the images, and further, why a particular treatment was selected) in the text regions 145A-n/links 148A-n.

In a further embodiment, presentation component 140 can utilize a language component 150 to present information, data, recommended treatments 112A-n on the screens 142A-n, whereby the language component 150 can utilize natural language processing (NLP) techniques (e.g., in processes 127A-n) to present information in a manner that can be readily understood by patient 102 and/or caregiver 103. Further, NLP enables the patient 102/caregiver 103 to interact (e.g., via questions and answers) with TRS 110 (e.g., via a chatbot/interface in HMI 186) in a manner particular to their language, dialect, sentence structure, vocabulary, phrasing, terminology, and suchlike, thus minimizing any frustration caregiver 103/patient 102 may have in interacting with an automated/computer system, e.g., TRS 110.

TRS 110 can further include an interaction component 152 configured to monitor interaction(s) 155A-n by either of the caregiver 103 and/or patient 102 with the respective information, images, etc., presented on recommendation screen 142A-n. For example, does caregiver 103 seek out further information regarding the recommended treatment 112A-n to confirm the recommended treatment 112A-n?, or is the recommended treatment 112A-n sufficiently accurate that the caregiver 103 has confidence that the recommended treatment 112A-n is applicable to patient 102's condition 101A-n? In the event of caregiver 103 undertakes a further extensive review of data/information presented on screen 142A-n, an inference can be made by the interaction component 152 (e.g., in conjunction with processes 127A-n) that the initially recommended treatment 112A-n is not entirely applicable and hence the respective data/steps the caregiver 103 uses/makes in interactions 155A-n can be used as part of retraining the multimodal model 122A-n, etc. In an aspect, functionality respectively provided by the language component 150 and interaction component 152 can combine to form a clinical guidance system (CGS).

A confirmation component 160 can also be included in TRS 110. In an embodiment, patient 102 can give their consent 165A-n to the recommended treatment 112A-n, whereby, per the knowledge imparted by implementing the various embodiments presented herein, the consent can be informed. In another embodiment, the confirmation component 160 can be configured to receive from the caregiver 103 a confirmation 165A-n that the recommended treatment 112A-n is to be implemented and/or consent given by patient 102.

In a further embodiment, information presented on the recommendation screen 142A-n can also be captured and/or printed in a report 149A-n, e.g., printed report, a digital report, etc., and can further include the interaction with the information presented on the recommendation screen 142A-n and whether confirmation/consent 165A-n was applied. Further, the multimodal component 120 can be configured to retrain the multimodal model 122A-n based on whether the recommendation 147A-n was selected, adjust the probability scores 146A-n, and the like, enabling the multimodal model 122A-n to be updated in accordance with the action of caregiver 103/patient 102.

An API component 170 can be configured to represent the respective components presented herein as APIs that can be shared/integrated with third party applications.

In an embodiment, one or more components (e.g., multimodal component 120, probability component 130, recommendation component 115, language component 150, interaction component 152, confirmation component 160, and suchlike) included in TRS 110 can utilize one or more processes 127A-n, wherein processes 127A-n can comprise various AI and ML technologies configured to review/analyze text, sentences, report data, images, metadata, and suchlike, in patient data 109A-n. As further described, any suitable/applicable AI/ML technologies can be utilized, e.g., processes 127A-n can include a large language model (LLM) for incorporation into the multimodal model 122A-n, wherein LLM technology can be configured to analyze content of patient data 109A-n to determine/recommend a treatment 112A-n.

It is to be appreciated that the various processes 127A-n and operations presented herein are simply examples of respective AI and ML operations and techniques, and any suitable AI/ML model/technology/technique/architecture can be utilized in accordance with the various embodiments presented herein. In an aspect, processes 127A-n can operate singly or in combination to create one or more applications configured to be implemented regarding identifying a particular medical condition 101A-n, e.g., a collection of processes 127A-n forming an application to identify issues relating to patient 102's medical condition 101A-n. Processes 127A-n can be based on application of terms, phrases, criteria, parameters, variables, and suchlike, in patient data 109A-n, recommended treatments 112A-n, interactions 155A-n, confirmations 165A-n, and suchlike. An example process 127A-n can include a vectoring technique such as bag of words (BOW) text vectors, and further, any suitable vectoring technology can be utilized, e.g., Euclidean distance, cosine similarity, etc. Other suitable AI/ML technologies/processes 127A-n that can be applied include, in a non-limiting list, any of vector representation via term frequency-inverse document frequency (tf-idf) capturing term/token frequency in any of patient data 109A-n, recommended treatments 112A-n, interactions 155A-n, confirmations 165A-n, and suchlike. Other applicable AI/ML technologies include, in a non-limiting list, neural network embedding, layer vector representation of terms/categories (e.g., common terms having different tense), bidirectional and auto-regressive transformer (BART) model architecture, a bidirectional encoder representation from transformers (BERT) model, a diffusion model, a variational autoencoder (VAE), a generative adversarial network (GAN), a language-based generative model such as a large language model (LLM), a generative pre-trained transformer (GPT), a long short-term memory (LSTM) network/operation, a sentence state LSTM (S-LSTM), a deep learning algorithm, a sequential neural network, a sequential neural network that enables persistent information, a recurrent neural network (RNN), a graph neural network, a convolutional neural network (CNN), a neural network, capsule network, a machine learning algorithm, a natural language processing (NLP) technique, sentiment analysis, bidirectional LSTM (BiLSTM), stacked BiLSTM, and suchlike. Accordingly, in an embodiment, implementation of multimodal component 120, probability component 130, recommendation component 115, presentation component 140, language component 150, interaction component 152, confirmation component 160, and suchlike, enables plain/natural language programming/annotation/correlation between any of patient data 109A-n, multimodal models 122A-n, probability models 132A-n, recommendations 112A-n, interactions 155A-n, confirmations 165A-n, and how any of the foregoing are presented on screens 142A-n and their subsequent interaction, e.g., by caregiver 103 and/or patient 102, to facilitate generation of recommendations 112A-n that readily pertain to patient 102′s condition 101A-n and further are implemented by caregiver 103 in their largely original form (e.g., recommendations 112A-n are sufficiently accurate and targeted that caregiver 103 only has to minimally change a recommendation 112A-n (as generated by recommendation component 115) and/or implements the recommendation 112A-n in it's as generated form), with caregiver 103 having sufficient confidence in the recommendation 112A-n generated by recommendation component 115.

Language models, LSTMs, BARTs, etc., can be formed with a neural network that is highly complex, for example, comprising billions of weighted parameters. Training of the language models, etc., can be conducted, e.g., by recommendation component 115 and other components of TRS 110, etc., with datasets, whereby the datasets can be formed using any suitable technology, such as data in patient data 109A-n, recommendations 112A-n, interactions 155A-n, confirmations 165A-n, information presented on screens 142A-n, hyperlinks 148A-n presented/selected on screens 142A-n, and suchlike. Further, as previously mentioned, patient data 109A-n, recommendations 112A-n, interactions 155A-n, confirmations 165A-n, information presented on screens 142A-n, hyperlinks 148A-n presented/selected on screens 142A-n, and suchlike, can comprise text, alphanumerics, numbers, single words, phrases, short statements, etc. Fine-tuning of a process 127A-n can comprise application of patient data 109A-n, recommendations 112A-n, interactions 155A-n, confirmations 165A-n, information presented on screens 142A-n, hyperlinks 148A-n presented/selected on screens 142A-n, and suchlike, to the process 127A-n, the process 127A-n is correspondingly adjusted by application of the historical data 107A-n, etc., such that, for example, weightings in the respective process 127-n are adjusted by application of the historical data 107A-n, and suchlike. As new information (e.g., interactions 155A-n captured and processed) historical data 107A-n can be updated accordingly, and further, processes 127A-n fine-tuned.

As further shown, TRS 110 can be communicatively coupled to a computer system 180. Computer system 180 can include a memory 184 that stores the respective computer executable components (e.g., multimodal component 120, probability component 130, recommendation component 115, presentation component 140, language component 150, interaction component 152, confirmation component 160, and suchlike) and further, a processor 182 configured to execute the computer executable components stored in the memory 184. Memory 184 can further be configured to store any of patient data 109A-n, recommendations 112A-n, scores 114A-n, interactions 155A-n, confirmations 165A-n, information presented on screens 142A-n, hyperlinks 148A-n presented/selected on screens 142A-n, processes 127A-n, AI similarity indexes S₁₋ₙ, AI vectors Vn, and suchlike. The computer system 180 can further include a human machine interface (HMI) 186 (e.g., a display, a graphical-user interface (GUI)) which can be configured to present various information including summary screens 142A-n, facilitate interactions 155A-n, mouse/cursor inputs, keyboard inputs, verbal inputs via a microphone, and suchlike. HMI 186 can include an interactive display/screen 187A-n to present the various recommendation screens 142A-n, patient data 109A-n, recommendations 112A-n, scores 114A-n, interactions 155A-n, confirmations 165A-n, hyperlinks 148A-n, processes 127A-n, AI similarity indexes SI-n, AI vectors Vn, and suchlike. Computer system 180 can further include an I/O component 188 to receive and/or transmit reports patient information 109A-n, questions/answers, hyperlinks 148A-n, and suchlike.

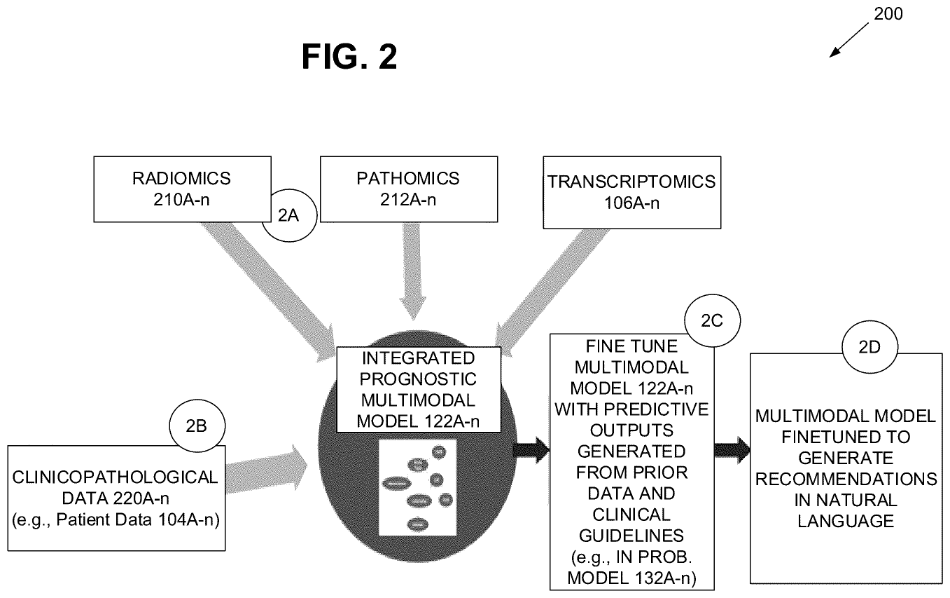

FIG. 2, schematic 200, illustrates an example process for generating one or more recommended treatments for a patient, in accordance with one or more embodiments. FIG. 2 is presented as a series of steps 2A-D.

At 2A, as previously mentioned with regard to FIG. 1, various data/information (e.g., in data 109A-n) can be utilized as part of construction of a multimodal model 122A-n, wherein such data can include radiomics 210A-n, pathomics 212A-n, and transcriptomics 106A-n, which can be combined by multimodal component 120 (in conjunction with processes 127A-n) to generate the multimodal model 122A-n. By utilizing a combination of radiomics 210A-n, pathomics 212A-n, and transcriptomics 106A-n enables generation of an integrated multimodal model 122A-n that comprises the features of a VLM having radiomic/pathomic functionality enhanced with transcriptomic functionality.

At 2B, clinic-pathological information 220A-n (e.g., in patient data 109A-n relating to/concerned with medical condition 101A-n signs and symptoms observable by caregiver 103 and/or laboratory examination results) can be further applied to the multimodal model 122A-n to enable the multimodal model 122A-n to be applied to generate one or more recommended treatments 112A-n.

At 2C, multimodal model 122A-n can be fine-tuned/trained with the feedback scores 114A-n generated by the probability component 130 and probability model 132A-n. E.g., on a first pass the multimodal model 122A-n generates a first recommendation 112X and a second recommendation 112Y, where recommendation treatments 112X and 112Y are deemed by the multimodal model 122A-n to have equal relevancy (or no relevancy determination has been performed). Application of recommendations 112X and 112Y to the probability model 132A-n leads to a generation of an applicability score of 75% for recommendation 112Y and 25% for recommendation 112X. Accordingly, recommendation 112Y is prioritized. Further, the probability knowledge can be subsequently applied to train/fine tune the multimodal model 122A-n such that on a subsequent implementation of multimodal model 122A-n under the same conditions 101A-n as experienced by patient 102, the multimodal model 122A-n can prioritize recommendation 112Y over 112X.

At 2D, NLP technology 127A-n can be further applied to the multimodal model 122A-n to facilitate understanding of the respective language, symbols, etc., utilized in patient data 109A-n. Further, NLP technology 127A-n can be utilized to enable interaction (e.g., interactions 155A-n) between a caregiver 103/patient 102 with TRS 110, such that, for example, when a question is asked by caregiver 103 regarding a recommended treatment 112A-n, the question can be intelligently processed (e.g., by any of language component 150, interaction component 152, recommendation component 115, probability component 130, multimodal component 120, and suchlike) enabling further information/context to be provided by TRS 110 and/or updating of the recommended treatments 112A-n, scores 114A-n, models 122A-n/132A-n, and suchlike.

Turning to FIG. 3, schematic 300 illustrates implementation of a probability model to further implement a recommendation, in accordance with an embodiment.

Schematic 300 illustrates a probability model 132A-n implemented in conjunction with a table of recommended treatments 112A-n generated by multimodal component 120 and multimodal model 122A-n.

As previously mentioned, probability model 132A can comprise nodes 320A-n connected by edges 330A-n. Nodes 320A-n can represent knowledge of patient 102′s condition 101A-n, medical/research knowledge regarding the medical condition 101A-n that patient 102 is suffering (e.g., in data 109A-n), wherein, in a simplified form, each node 320A-n can include a single input and a pair of outputs, whereby each output can have a particular probability. As shown, nodes 320A, 320B, 320C, and 320D indicate a flow of knowledge, however, knowledge regarding node 320E is incomplete. By applying known information (e.g., in historical data 107A-n) it is possible to determine, with an acceptable degree of confidence, data to apply at 320E (e.g., as brought in from historical data 107A-n). Hence, per the upper portion of probability model 132A, it can be determined that recommended treatment 112C most pertains to patient 102's condition 101A-n (as node 320C has an associated probability of 80%), and should be the treatment 112A-n reviewed first by caregiver 103.

In an aspect, the resulting model presented in FIG. 3 can be considered to be a combination/amalgamation of the multimodal model 122A-n and the probability model 132A-n, such that the nodes 320A-n are derived from the information provided to the multimodal model 122A-n and the edges are probabilities derived from the probability model 132A-n.

In an embodiment, data for a missing modality or subject (e.g., at node 320E) can be simulated using any suitable technology, such as a diffusion model, a generative adversarial network, population statistics, and the like, for data imputations and/or to increase data variability/heterogeneity to improve performance of multimodal model 122A-n.

Per the lower portion of schematic 300, while nodes 320F-I represent knowledge, the knowledge is not applicable to the medical condition 101A-n of patient 102, accordingly, any recommended treatments (e.g., treatments 112E-I) generated by nodes 320F-I can be ignored. In an aspect, treatments 112E-I may pertain to treatment of patient 102, but, for example, local medical guidelines/procedures prevents implementation of 112E-I. In an embodiment, the guideline can be updated in accordance with results provided by model 132A-n.

In an example of implementation of the multimodal model 122A-n, an example respective decision being configured for node 320A, node 320A can comprise the decision of is patient 102 male or female, such that with patient 102 being female, and the upper portion of multimodal model 122A pertaining to female-related illness, navigation of the multimodal model 122A advances to node 320B, while the lower portion of multimodal model 122A pertains to the male-related illness and hence, navigation of the multimodal model 122A does not occur for the lower portion.

Further, regarding the respective percentages/probabilities establishing navigation through the multimodal model 122A, e.g., from node 320E to node 320D (has a probability derived from population statistics) of 80%, from node 320E to node 320C (has a probability derived from population statistics) of 20%.

As further shown, multimodal model 122A can further include a mobility node 3201, wherein the node 320I can represent a treatment (e.g., provided by a physician, etc.) that departs from standard/established treatment guidelines/protocols, but the outcome/study of the treatment can be captured in the historical data 107A-n and applied as a node, e.g., mobility node 320I. Accordingly, multimodal model 122A can include nodes representing established medical protocols and also treatments that may represent a generally studied/recommended treatment, per node 320I. For example, a treatment protocol may be too toxic for patient 102, and hence, an alternative treatment 320I can be applied that is less toxic to the patient 102.

### Methods

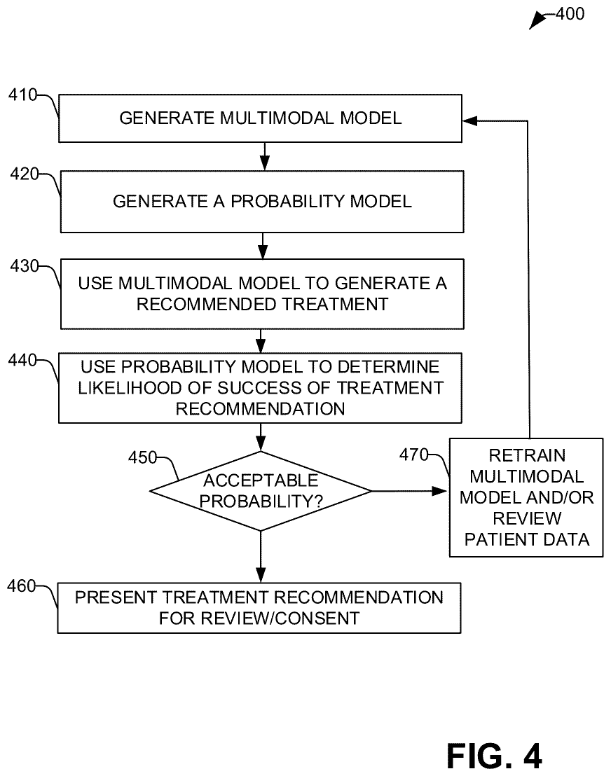

FIG. 4, via flow-chart 400, presents an example computer-implemented method for generating a recommended treatment for a patient's condition, in accordance with an embodiment.

At 410, a multimodal model (e.g., model 122A-n) can be generated (e.g., by multimodal component 120), and configured to comprise of pathomics, radiomics, transcriptomics, and suchlike. The multimodal model can be constructed based on prior knowledge (e.g., in pre-existing imaging, measurements, diagnostics, etc.) in conjunction with information regarding a patient's condition (e.g., medical condition 101A-n and information/data pertaining thereto in patient 102's medical information).

At 420, a probability model (e.g., model 132A-n) can be generated (e.g., by probability component 130). The probability model can be configured based on application of particular treatments for a medical condition (e.g., a medical condition 101A-n), wherein the particular treatments can be assessed based on the success of their implementation per historical data (e.g., historical data 107A-n) and a previous situation having conditions similar/comparable to the medical condition being experienced by patient 102.

At 430, the multimodal model can be utilized to generate a recommended treatment based on analysis of the patient's information and the prior knowledge. In an embodiment, a series of recommended treatments can be generated by the multimodal model.

At 440, the probability model can be utilized to assess respective prior treatments applicable to the patient's condition, and provide a measure (e.g., score 114A-n) of their applicability to the patient's condition. The respective identified prior treatments and the associated measure can be applied to the recommended treatments generated by the multimodal model.

At 450, a determination can be made (e.g., by recommendation component 115 in conjunction with processes 127A-n) regarding whether a recommended treatment generated by the multimodal model has an acceptable level of confidence/score regarding applicability of the recommended treatment for the patient's condition. In response to a determination that a recommended treatment has an acceptable level of probability of success, methodology 400 can advance to step 460, whereby the recommended treatment can be presented (e.g., on screen 142A-n) in conjunction with the probability/confidence score, and any associated images, data, text, and suchlike.

At 450, in response to a determination that one or more recommended treatments do not have an acceptable level of confidence regarding implementation on the patient, methodology 400 can advance to step 470, whereupon the model(s) (e.g., model(s) 122A-n/132A-n) can be retrained in accordance with one or more findings as to why the recommended treatment(s) had an unacceptable level of confidence even though the model(s) considered the recommended treatment(s) to be viable. Further, patient information 109A-n can be reviewed to determine if the patient is suffering an unexpected/novel condition.

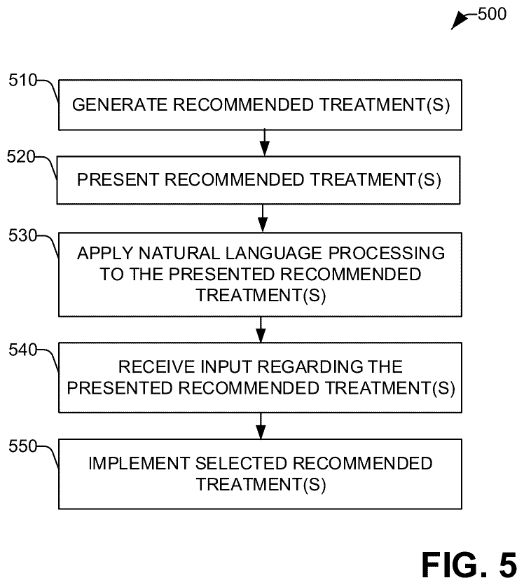

FIG. 5, via flowchart 500, presents an example computer-implemented method for implementing a recommended treatment for a patient's condition, in accordance with an embodiment.

At 510, one or more recommended treatments (e.g., recommended treatment 112A-n) can be generated (e.g., by recommendation component 115). In an embodiment, each of the recommended treatments can have an associated score (e.g., score 114A-n) of confidence/applicability regarding the respective recommended treatment in treating the patient's condition (e.g., medical condition 101A-n of patient 102).

At 520, the one or more recommended treatments can be presented on a screen (e.g., screen 142A-n). The one or more recommended treatments can be presented in conjunction with the probability/confidence score, and any associated images, data, text, and suchlike (e.g., patient data 109A-n).

At 530, to enable understanding of the information presented on the screen, NLP technology can be applied to the information, enabling the patient and/or caregiver (e.g., caregiver 103) to interact with the presented information.

At 540, the screen can be part of an interactive display (e.g., screen 187A-n of HMI 186) comprising any/all of mouse, keyboard, microphone, and any other suitable means for interaction. In an example scenario of interaction, caregiver can review the presented information to ensure that the recommended treatment is suitable/effective. Accordingly, the caregiver can navigate (e.g., via interactions 155A-n) through the information, e.g., via verbal commands, hyperlink selection, and suchlike. In an embodiment, the caregiver can adjust/modify the recommended treatment (e.g., an initial recommended treatment) generated by the treatment recommendation system (e.g., TRS 110), such that the recommended treatment can be updated in accordance with the caregiver's instruction, generating a modified recommended treatment. The interactions can be monitored/recorded by an interaction component (e.g., interaction component 152) and saved (e.g., as historical data 107A-n) to further train/fine-tune the multimodal model (e.g., multimodal model 122A-n) and/or the probability model (e.g., probability model 132A-n).

At 550, an input (e.g., confirmation 165A-n) can be received (e.g., by confirmation component 160) regarding selection/confirmation of implementing the recommended treatment (e.g., original recommended treatment or modified recommended treatment). In response to the confirmation input, the recommended treatment can be implemented (e.g., with the patient's consent).

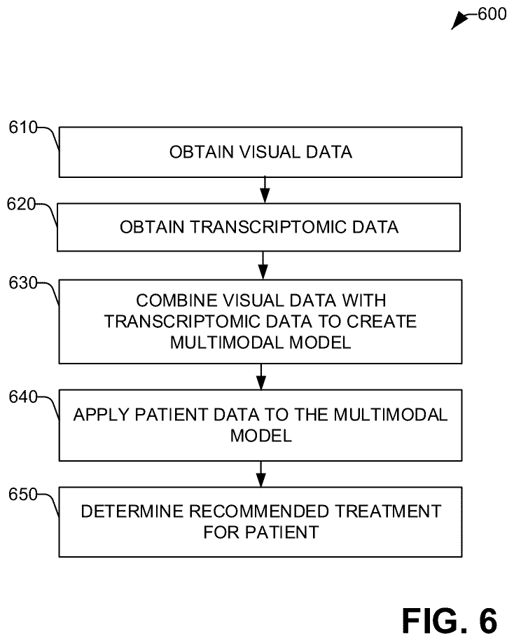

FIG. 6, via flowchart 600, presents an example computer-implemented method for generating a VLM incorporating radiomics, pathomics, transcriptomics, and suchlike, in accordance with an embodiment.

At 610, visual data (e.g., VLM 105A-n) can be received at a system (e.g., TRS 110), wherein the visual data can be a VLM for radiomics and pathomics processing and analysis.

At 620, transcriptomic data (e.g., transcriptomic data 106A-n) can be received (e.g., at TRS 110).

At 630, the visual data can be combined (e.g., by multimodal component 120) with the transcriptomic data to create a multimodal model (e.g., multimodal model 122A-n).

At 640, patient data (e.g., patient data 109A-n) can be applied (e.g., by multimodal component 120) to the multimodal model.

At 650, one or more recommended treatments (e.g., recommended treatments 112A-n) for the patient can be determined from the multimodal model as a function of applying the patient data to the multimodal model. As previously mentioned, the one or more recommended treatments can be applied to a probability model to enable confirmation of the one or more recommended treatments are applicable to the patient's condition (e.g., medical condition 101A-n). For example, a risk score (e.g., score 114A-n) is generated for the recommended treatment(s) enabling a confidence of applicability of the recommended treatment to the patient's condition.

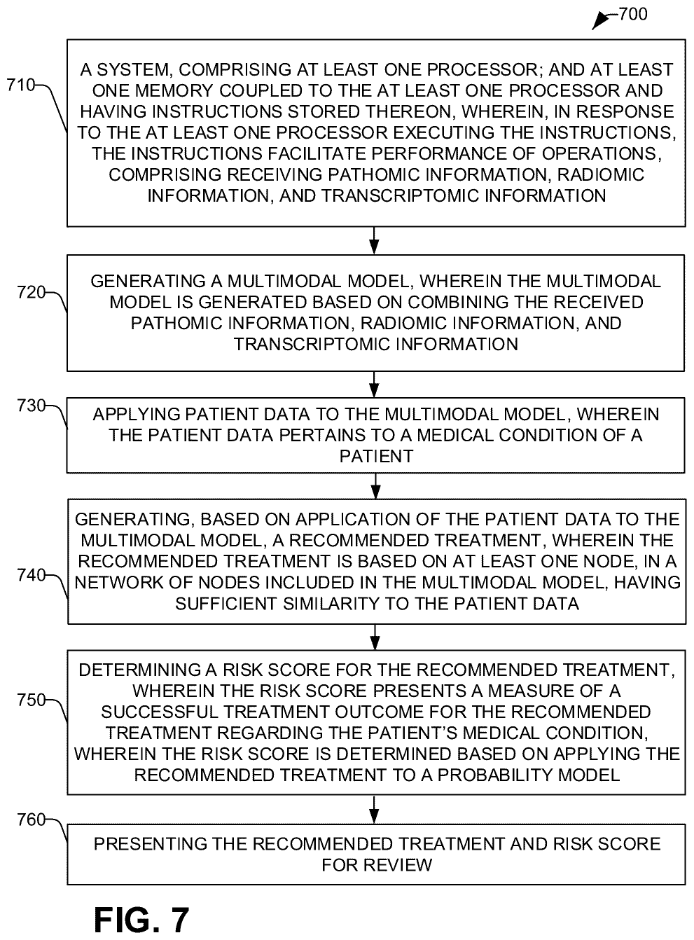

FIG. 7, via flowchart 700, presents an example computer-implemented method for implementing a multimodal model in conjunction with a probability model to identify a medical condition and further identify/recommend a treatment, in accordance with an embodiment. At 710, the method 700 can be implemented by a system (e.g., TRS 110), comprising: at least one processor (e.g., processor 182A-n), and a memory (e.g., memory 184) coupled to the at least one processor and having instructions stored thereon, wherein, in response to the at least one processor executing the instructions, the instructions facilitate performance of operations, comprising: receiving pathomic information (e.g., radiomic information 212A-n), radiomic information (e.g., radiomic information 210A-n), and transcriptomic information (e.g., transcriptomic information 106A-n).

At 720, method 700 can further comprise generating a multimodal model (e.g., multimodal model 120), wherein the multimodal model is generated based on combining the received pathomic information, radiomic information, and transcriptomic information.

At 730, method 700 can further comprise applying patient data (e.g., patient data 109A-n) to the multimodal model, wherein the patient data pertains to a medical condition of a patient.

At 740, method 700 can further comprise generating, based on application of the patient data to the multimodal model, a recommended treatment (e.g., treatment 112A-n), wherein the recommended treatment is based on at least one node (e.g., nodes 320A-n), in a network of nodes included in the multimodal model, having sufficient similarity to the patient data.

At 750, method 700 can further comprise determining a risk score for the recommended treatment, wherein the risk score presents a measure of a successful treatment outcome for the recommended treatment regarding the patient's medical condition, wherein the risk score is determined based on applying the recommended treatment to a probability model.

At 760, method 700 can further comprise presenting the recommended treatment and risk score for review.

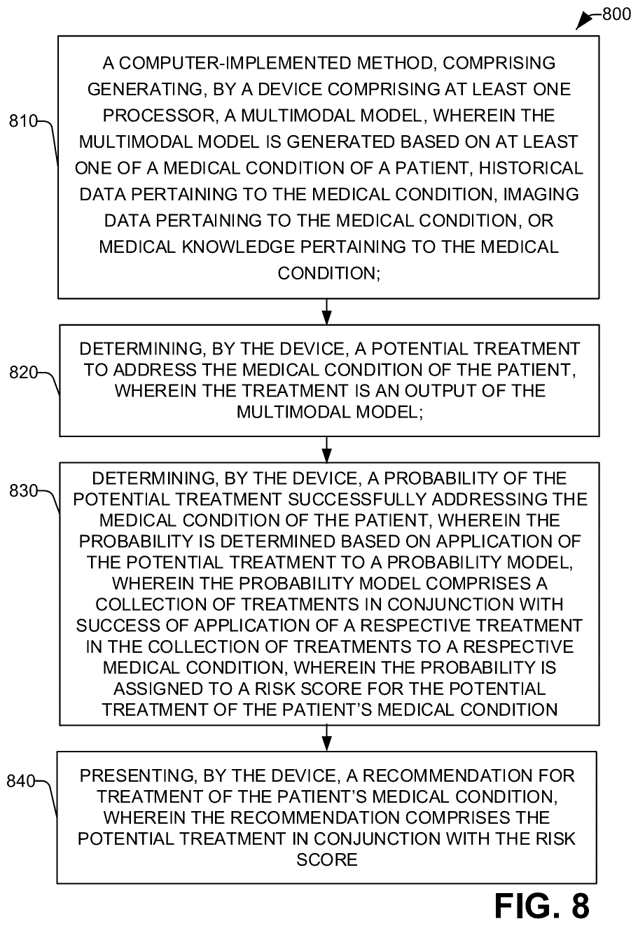

FIG. 8, via flowchart 800, presents an example computer-implemented method for utilizing a multimodal model in conjunction with a probability model to identify a medical condition and further identify/recommend a treatment, in accordance with an embodiment. At 810, the method 800 can comprise generating, by a device comprising at least one processor, a multimodal model, wherein the multimodal model is generated based on at least one of a medical condition of a patient, historical data pertaining to the medical condition, imaging data pertaining to the medical condition, or medical knowledge pertaining to the medical condition.

At 820, method 800 can further comprise determining, by the device, a potential treatment to address the medical condition of the patient, wherein the treatment is an output of the multimodal model.

At 830, method 800 can further comprise determining, by the device, a probability of the potential treatment successfully addressing the medical condition of the patient, wherein the probability is determined based on application of the potential treatment to a probability model, wherein the probability model comprises a collection of treatments in conjunction with success of application of a respective treatment in the collection of treatments to a respective medical condition, wherein the probability is assigned to a risk score for the potential treatment of the patient's medical condition.

At 840, method 800 can further comprise presenting, by the device, a recommendation for treatment of the patient's medical condition, wherein the recommendation comprises the potential treatment in conjunction with the risk score.

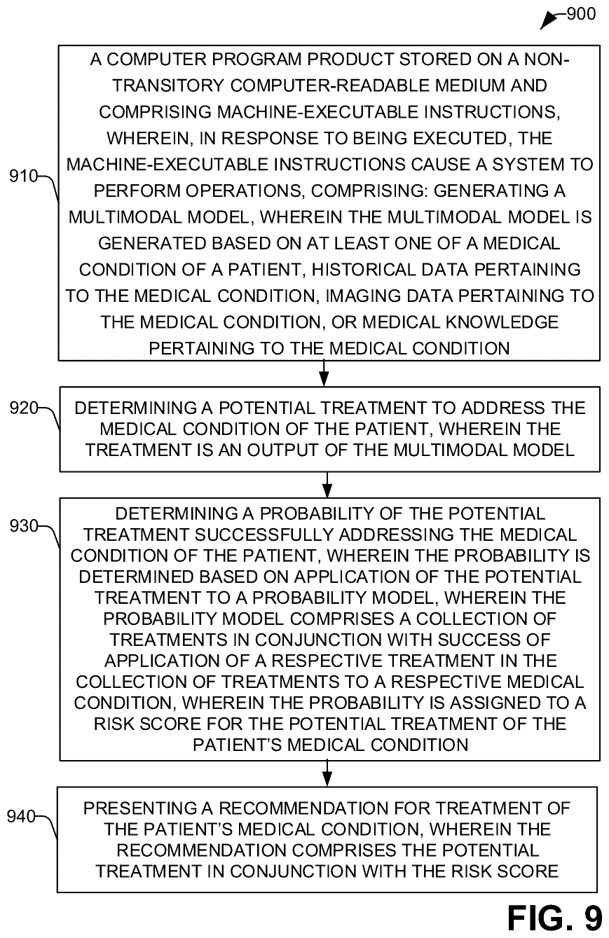

FIG. 9, via flowchart 900, presents an example computer-implemented method for utilizing a multimodal model in conjunction with a probability model to identify a medical condition and further identify/recommend a treatment, in accordance with an embodiment. At 910, the method 900 can be performed by a computer program product stored on a non-transitory computer-readable medium and comprising machine-executable instructions, wherein, in response to being executed, the machine-executable instructions cause a system to perform operations, comprising generating a multimodal model, wherein the multimodal model is generated based on at least one of a medical condition of a patient, historical data pertaining to the medical condition, imaging data pertaining to the medical condition, or medical knowledge pertaining to the medical condition.

At 920, method 900 can further comprise determining a potential treatment to address the medical condition of the patient, wherein the treatment is an output of the multimodal model.

At 930, method 900 can further comprise determining a probability of the potential treatment successfully addressing the medical condition of the patient, wherein the probability is determined based on application of the potential treatment to a probability model, wherein the probability model comprises a collection of treatments in conjunction with success of application of a respective treatment in the collection of treatments to a respective medical condition, wherein the probability is assigned to a risk score for the potential treatment of the patient's medical condition.

At 940, method 900 can further comprise presenting a recommendation for

treatment of the patient's medical condition, wherein the recommendation comprises the potential treatment in conjunction with the risk score.

As used herein, the terms “infer”, “inference”, “determine”, and suchlike, refer generally to the process of reasoning about or inferring states of the system, environment, and/or user from a set of observations as captured via events and/or data. Inference can be employed to identify a specific context or action, or can generate a probability distribution over states, for example. The inference can be probabilistic-that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources.

Per the various embodiments presented herein, various components included in TRS 110, recommendation component 115, multimodal component 120, probability component 130, presentation component 140, interaction component 152, language component 150, confirmation component 160, API component 170, and suchlike, can include AI/ML and reasoning techniques and technologies (e.g., processes 127A-n) that employ probabilistic and/or statistical-based analysis to prognose or infer an action that a user desires to be automatically performed. The various embodiments presented herein can utilize various ML-based schemes for carrying out various aspects thereof. For example, a process 127A-n (e.g., by multimodal component 120) to automatically generate the multimodal model 122A-n and the recommended treatment 112A-n; a process 127A-n (e.g., by probability component 130) to automatically generate the probability model 132A-n and further assess viability of the recommended treatment 112A-n; a process 127A-n (e.g., by recommendation component 115/presentation component 140) to automatically present the recommended treatment 112A-n for review; a process 127A-n (e.g., by interaction component 152) for automatically implementing natural language processing to enable interaction with presented information (e.g., on screen 142A-n); a process 127A-n (e.g., by confirmation component 160) to automatically receive an indication (e.g., consent/confirmation 165A-n) of acceptance of the recommended treatment; a process 127A-n (e.g., by any of interaction component 152, multimodal component 120, probability component 130, recommendation component 115, and/or interaction component 152) to record interaction by patient 102 and/or caregiver 103 adjusting a recommended treatment and/or reviewing further information presented on screen 142A-n, and further applying the interaction/adjustment to train/fine-tune a multimodal model 122A-n and/or probability model 132A-n, and suchlike, as previously mentioned herein, can be facilitated via an automatic classifier system and process.

A classifier is a function that maps an input attribute vector, x=(x1, x2, x3, x4, xn), to a class label class(x). The classifier can also output a confidence that the input belongs to a class, that is, f(x)=confidence(class(x)). Such classification can employ a probabilistic and/or statistical-based analysis (e.g., factoring into the analysis utilities and costs) to prognose or infer an action that a user desires to be automatically performed (e.g., automatically identifying and presenting a recommended treatment 112A-n, generating an applicability score 114A-n, interaction with screen 142A-n with natural language processing, and operations related thereto).

A support vector machine (SVM) is an example of a classifier that can be employed. The SVM operates by finding a hypersurface in the space of possible inputs that splits the triggering input events from the non-triggering events in an optimal way. Intuitively, this makes the classification correct for testing data that is near, but not identical to training data. Other directed and undirected model classification approaches include, e.g., naïve Bayes, Bayesian networks, a random forest, decision trees, knowledge graphs, neural networks, fuzzy logic models, and probabilistic classification models providing different patterns of independence can be employed. Classification as used herein is inclusive of statistical regression that is utilized to develop models of priority.

As will be readily appreciated from the subject specification, the various embodiments can employ classifiers that are explicitly trained (e.g., via a generic training data) as well as implicitly trained (e.g., via observing user behavior, receiving extrinsic information). For example, SVM's are configured via a learning or training phase within a classifier constructor and feature selection module. Thus, the classifier(s) can be used to automatically learn and perform a number of functions, including but not limited to determining according to predetermined criteria, content in patient data 109A-n to create multimodal models 122A-n, probability models 132A-n, recommended treatments 112A-n, scores 114A-n, and suchlike, for example.

As described supra, inferences can be made, and automated operations performed, based on numerous pieces of information. For example, whether sufficient information/context is available to infer, with a high degree of confidence, a treatment 112A-n is applicable to patient 102's condition 101A-n, focus of attention of an interaction 155A-n with information on screen 142A-n, and suchlike, to enable a patient 102/caregiver 103 to readily determine a treatment 112A-n to utilize for patient 102's medical condition.

### Example Applications and Use

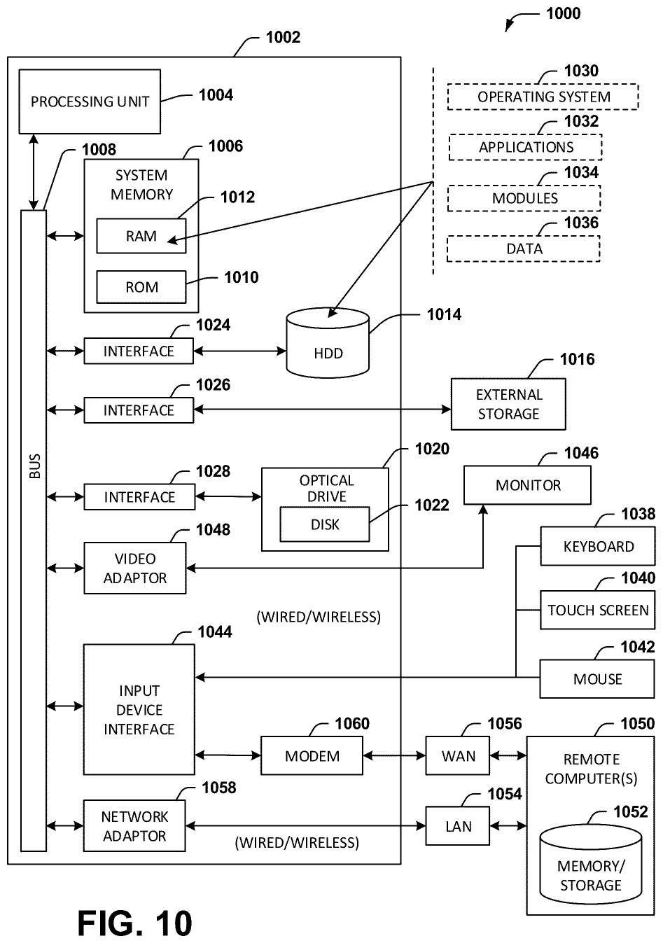

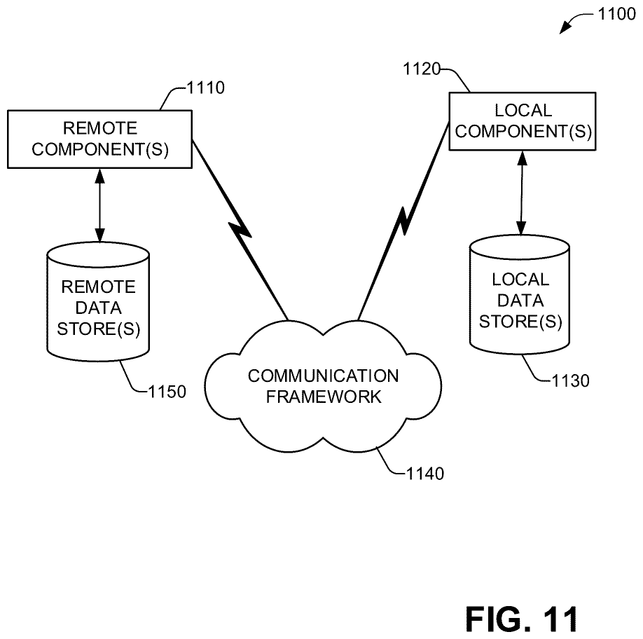

Turning next to FIGS. 10 and 11, a detailed description is provided of additional context for the one or more embodiments described herein with FIGS. 1-9.

In order to provide additional context for various embodiments described herein, FIG. 10 and the following discussion are intended to provide a brief, general description of a suitable computing environment 1000 in which the various embodiments described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the methods can be practiced with other computer system configurations, including single-processor or multiprocessor computer systems, minicomputers, mainframe computers, IoT devices, distributed computing systems, as well as personal computers, hand-held computing devices, microprocessor-based or programmable consumer electronics, and the like, each of which can be operatively coupled to one or more associated devices.

The embodiments illustrated herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 10, the example environment 1000 for implementing various embodiments of the aspects described herein includes a computer 1002, the computer 1002 including a processing unit 1004, a system memory 1006 and a system bus 1008. The system bus 1008 couples system components including, but not limited to, the system memory 1006 to the processing unit 1004. The processing unit 1004 can be any of various commercially available processors and may include a cache memory. Dual microprocessors and other multi-processor architectures can also be employed as the processing unit 1004.

The system bus 1008 can be any of several types of bus structure that can further interconnect to a memory bus (with or without a memory controller), a peripheral bus, and a local bus using any of a variety of commercially available bus architectures. The system memory 1006 includes ROM 1010 and RAM 1012. A basic input/output system (BIOS) can be stored in a non-volatile memory such as ROM, erasable programmable read only memory (EPROM), EEPROM, which BIOS contains the basic routines that help to transfer information between elements within the computer 1002, such as during startup. The RAM 1012 can also include a high-speed RAM such as static RAM for caching data.

The computer 1002 further includes an internal hard disk drive (HDD) 1014 (e.g., EIDE, SATA), one or more external storage devices 1016 (e.g., a magnetic floppy disk drive (FDD) 1016, a memory stick or flash drive reader, a memory card reader, etc.) and an optical disk drive 1020 (e.g., which can read or write from a CD-ROM disc, a DVD, a BD, etc.). While the internal HDD 1014 is illustrated as located within the computer 1002, the internal HDD 1014 can also be configured for external use in a suitable chassis (not shown). Additionally, while not shown in environment 1000, a solid-state drive (SSD) could be used in addition to, or in place of, an HDD 1014. The HDD 1014, external storage device(s) 1016 and optical disk drive 1022 can be connected to the system bus 1008 by an HDD interface 1024, an external storage interface 1026 and an optical drive interface 1028, respectively. The interface 1024 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1002, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1012, including an operating system 1030, one or more application programs 1032, other program modules 1034 and program data 1036. All or portions of the operating system, applications, modules, and/or data can also be cached in the RAM 1012. The systems and methods described herein can be implemented utilizing various commercially available operating systems or combinations of operating systems.

Computer 1002 can optionally comprise emulation technologies. For example, a hypervisor (not shown) or other intermediary can emulate a hardware environment for operating system 1030, and the emulated hardware can optionally be different from the hardware illustrated in FIG. 10. In such an embodiment, operating system 1030 can comprise one virtual machine (VM) of multiple VMs hosted at computer 1002. Furthermore, operating system 1030 can provide runtime environments, such as the Java runtime environment or the .NET framework, for applications 1032. Runtime environments are consistent execution environments that allow applications 1032 to run on any operating system that includes the runtime environment. Similarly, operating system 1030 can support containers, and applications 1032 can be in the form of containers, which are lightweight, standalone, executable packages of software that include, e.g., code, runtime, system tools, system libraries and settings for an application.

Further, computer 1002 can comprise a security module, such as a trusted processing module (TPM). For instance with a TPM, boot components hash next in time boot components, and wait for a match of results to secured values, before loading a next boot component. This process can take place at any layer in the code execution stack of computer 1002, e.g., applied at the application execution level or at the operating system (OS) kernel level, thereby enabling security at any level of code execution.

A user can enter commands and information into the computer 1002 through one or more wired/wireless input devices, e.g., a keyboard 1038, a touch screen 1040, and a pointing device, such as a mouse 1042. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1004 through an input device interface 1044 that can be coupled to the system bus 1008, but can be connected by other interfaces, such as a parallel port, an IEEE 1394 serial port, a game port, a USB port, an IR interface, a BLUETOOTH® interface, etc.

A monitor 1046 or other type of display device can be also connected to the system bus 1008 via an interface, such as a video adapter 1048. In addition to the monitor 1046, a computer typically includes other peripheral output devices (not shown), such as speakers, printers, etc.

The computer 1002 can operate in a networked environment using logical connections via wired and/or wireless communications to one or more remote computers, such as a remote computer(s) 1050. The remote computer(s) 1050 can be a workstation, a server computer, a router, a personal computer, portable computer, microprocessor-based entertainment appliance, a peer device or other common network node, and typically includes many or all of the elements described relative to the computer 1002, although, for purposes of brevity, only a memory/storage device 1052 is illustrated. The logical connections depicted include wired/wireless connectivity to a local area network (LAN) 1054 and/or larger networks, e.g., a wide area network (WAN) 1056. Such LAN and WAN networking environments are commonplace in offices and companies, and facilitate enterprise-wide computer networks, such as intranets, all of which can connect to a global communications network, e.g., the internet.

When used in a LAN networking environment, the computer 1002 can be connected to the local network 1054 through a wired and/or wireless communication network interface or adapter 1058. The adapter 1058 can facilitate wired or wireless communication to the LAN 1054, which can also include a wireless access point (AP) disposed thereon for communicating with the adapter 1058 in a wireless mode.

When used in a WAN networking environment, the computer 1002 can include a modem 1060 or can be connected to a communications server on the WAN 1056 via other means for establishing communications over the WAN 1056, such as by way of the internet. The modem 1060, which can be internal or external and a wired or wireless device, can be connected to the system bus 1008 via the input device interface 1044. In a networked environment, program modules depicted relative to the computer 1002 or portions thereof, can be stored in the remote memory/storage device 1052. It will be appreciated that the network connections shown are example and other means of establishing a communications link between the computers can be used.

When used in either a LAN or WAN networking environment, the computer 1002 can access cloud storage systems or other network-based storage systems in addition to, or in place of, external storage devices 1016 as described above. Generally, a connection between the computer 1002 and a cloud storage system can be established over a LAN 1054 or WAN 1056 e.g., by the adapter 1058 or modem 1060, respectively. Upon connecting the computer 1002 to an associated cloud storage system, the external storage interface 1026 can, with the aid of the adapter 1058 and/or modem 1060, manage storage provided by the cloud storage system as it would other types of external storage. For instance, the external storage interface 1026 can be configured to provide access to cloud storage sources as if those sources were physically connected to the computer 1002.

The computer 1002 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

The above description includes non-limiting examples of the various embodiments. It is, of course, not possible to describe every conceivable combination of components or methodologies for purposes of describing the disclosed subject matter, and one skilled in the art may recognize that further combinations and permutations of the various embodiments are possible. The disclosed subject matter is intended to embrace all such alterations, modifications, and variations that fall within the spirit and scope of the appended claims.

Referring now to details of one or more elements illustrated at FIG. 11, an illustrative cloud computing environment 1100 is depicted. FIG. 11 is a schematic block diagram of a computing environment 1100 with which the disclosed subject matter can interact. The system 1100 comprises one or more remote component(s) 1110. The remote component(s) 1110 can be hardware and/or software (e.g., threads, processes, computing devices). In some embodiments, remote component(s) 1110 can be a distributed computer system, connected to a local automatic scaling component and/or programs that use the resources of a distributed computer system, via communication framework 1140. Communication framework 1140 can comprise wired network devices, wireless network devices, mobile devices, wearable devices, radio access network devices, gateway devices, femtocell devices, servers, etc.

The system 1100 also comprises one or more local component(s) 1120. The local component(s) 1120 can be hardware and/or software (e.g., threads, processes, computing devices). In some embodiments, local component(s) 1120 can comprise an automatic scaling component and/or programs that communicate/use the remote resources 1110 and 1120, etc., connected to a remotely located distributed computing system via communication framework 1140.

One possible communication between a remote component(s) 1110 and a local component(s) 1120 can be in the form of a data packet adapted to be transmitted between two or more computer processes. Another possible communication between a remote component(s) 1110 and a local component(s) 1120 can be in the form of circuit-switched data adapted to be transmitted between two or more computer processes in radio time slots. The system 1100 comprises a communication framework 1140 that can be employed to facilitate communications between the remote component(s) 1110 and the local component(s) 1120, and can comprise an air interface, e.g., Uu interface of a UMTS network, via a long-term evolution (LTE) network, etc. Remote component(s) 1110 can be operably connected to one or more remote data store(s) 1150, such as a hard drive, solid state drive, SIM card, device memory, etc., that can be employed to store information on the remote component(s) 1110 side of communication framework 1140. Similarly, local component(s) 1120 can be operably connected to one or more local data store(s) 1130, that can be employed to store information on the local component(s) 1120 side of communication framework 1140.

With regard to the various functions performed by the above described components, devices, circuits, systems, etc., the terms (including a reference to a “means”) used to describe such components are intended to also include, unless otherwise indicated, any structure(s) which performs the specified function of the described component (e.g., a functional equivalent), even if not structurally equivalent to the disclosed structure. In addition, while a particular feature of the disclosed subject matter may have been disclosed with respect to only one of several implementations, such feature may be combined with one or more other features of the other implementations as may be desired and advantageous for any given or particular application.

The terms “exemplary” and/or “demonstrative” as used herein are intended to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent structures and techniques known to one skilled in the art. Furthermore, to the extent that the terms “includes,” “has,” “contains,” and other similar words are used in either the detailed description or the claims, such terms are intended to be inclusive-in a manner similar to the term “comprising” as an open transition word-without precluding any additional or other elements.

The term “or” as used herein is intended to mean an inclusive “or” rather than an exclusive “or.” For example, the phrase “A or B” is intended to include instances of A, B, and both A and B. Additionally, the articles “a” and “an” as used in this application and the appended claims should generally be construed to mean “one or more” unless either otherwise specified or clear from the context to be directed to a singular form.

The term “set” as employed herein excludes the empty set, i.e., the set with no elements therein. Thus, a “set” in the subject disclosure includes one or more elements or entities. Likewise, the term “group” as utilized herein refers to a collection of one or more entities.

The terms “first,” “second,” “third,” and so forth, as used in the claims, unless otherwise clear by context, is for clarity only and doesn't otherwise indicate or imply any order in time. For instance, “a first determination,” “a second determination,” and “a third determination,” does not indicate or imply that the first determination is to be made before the second determination, or vice versa, etc.

As used in this disclosure, in some embodiments, the terms “component,” “system” and the like are intended to refer to, or comprise, a computer-related entity or an entity related to an operational apparatus with one or more specific functionalities, wherein the entity can be either hardware, a combination of hardware and software, software, or software in execution. As an example, a component can be, but is not limited to being, a process running on a processor, a processor, an object, an executable, a thread of execution, computer-executable instructions, a program, and/or a computer. By way of illustration and not limitation, both an application running on a server and the server can be a component.

One or more components can reside within a process and/or thread of execution and a component can be localized on one computer and/or distributed between two or more computers. In addition, these components can execute from various computer readable media having various data structures stored thereon. The components can communicate via local and/or remote processes such as in accordance with a signal having one or more data packets (e.g., data from one component interacting with another component in a local system, distributed system, and/or across a network such as the internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software or firmware that confers at least in part the functionality of the electronic components. While various components have been illustrated as separate components, it will be appreciated that multiple components can be implemented as a single component, or a single component can be implemented as multiple components, without departing from example embodiments.

The term “facilitate” as used herein is in the context of a system, device or component “facilitating” one or more actions or operations, in respect of the nature of complex computing environments in which multiple components and/or multiple devices can be involved in some computing operations. Non-limiting examples of actions that may or may not involve multiple components and/or multiple devices comprise transmitting or receiving data, establishing a connection between devices, determining intermediate results toward obtaining a result, etc. In this regard, a computing device or component can facilitate an operation by playing any part in accomplishing the operation. When operations of a component are described herein, it is thus to be understood that where the operations are described as facilitated by the component, the operations can be optionally completed with the cooperation of one or more other computing devices or components, such as, but not limited to, sensors, antennae, audio and/or visual output devices, other devices, etc.

Further, the various embodiments can be implemented as a method, apparatus or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable (or machine-readable) device or computer-readable (or machine-readable) storage/communications media. For example, computer readable storage media can comprise, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

Moreover, terms such as “mobile device equipment,” “mobile station,” “mobile,” “subscriber station,” “access terminal,” “terminal,” “handset,” “communication device,” “mobile device” (and/or terms representing similar terminology) can refer to a wireless device utilized by a subscriber or mobile device of a wireless communication service to receive or convey data, control, voice, video, sound, gaming or substantially any data-stream or signaling-stream. The foregoing terms are utilized interchangeably herein and with reference to the related drawings. Likewise, the terms “access point (AP),” “Base Station (BS),” “BS transceiver,” “BS device,” “cell site,” “cell site device,” “gNode B (gNB),” “evolved Node B (eNode B, eNB),” “home Node B (HNB)” and the like, refer to wireless network components or appliances that transmit and/or receive data, control, voice, video, sound, gaming or substantially any data-stream or signaling-stream from one or more subscriber stations. Data and signaling streams can be packetized or frame-based flows.

Furthermore, the terms “device,” “communication device,” “mobile device,” “subscriber,” “client entity,” “consumer,” “client entity,” “entity” and the like are employed interchangeably throughout, unless context warrants particular distinctions among the terms. It should be appreciated that such terms can refer to human entities or automated components supported through artificial intelligence (e.g., a capacity to make inference based on complex mathematical formalisms), which can provide simulated vision, sound recognition and so forth.

It should be noted that although various aspects and embodiments are described herein in the context of 5G or other next generation networks, the disclosed aspects are not limited to a 5G implementation, and can be applied in other network next generation implementations, such as sixth generation (6G), or other wireless systems. In this regard, aspects or features of the disclosed embodiments can be exploited in substantially any wireless communication technology. Such wireless communication technologies can include universal mobile telecommunications system (UMTS), global system for mobile communication (GSM), code division multiple access (CDMA), wideband CDMA (WCMDA), CDMA2000, time division multiple access (TDMA), frequency division multiple access (FDMA), multi-carrier CDMA (MC-CDMA), single-carrier CDMA (SC-CDMA), single-carrier FDMA (SC-FDMA), orthogonal frequency division multiplexing (OFDM), discrete Fourier transform spread OFDM (DFT-spread OFDM), filter bank based multi-carrier (FBMC), zero tail DFT-spread-OFDM (ZT DFT-s-OFDM), generalized frequency division multiplexing (GFDM), fixed mobile convergence (FMC), universal fixed mobile convergence (UFMC), unique word OFDM (UW-OFDM), unique word DFT-spread OFDM (UW DFT-Spread-OFDM), cyclic prefix OFDM (CP-OFDM), resource-block-filtered OFDM, wireless fidelity (Wi-Fi), worldwide interoperability for microwave access (WiMAX), wireless local area network (WLAN), general packet radio service (GPRS), enhanced GPRS, third generation partnership project (3GPP), long term evolution (LTE), 5G, third generation partnership project 2 (3GPP2), ultra-mobile broadband (UMB), high speed packet access (HSPA), evolved high speed packet access (HSPA+), high-speed downlink packet access (HSDPA), high-speed uplink packet access (HSUPA), Zigbee, or another institute of electrical and electronics engineers (IEEE) 802.12 technology.

It is to be understood that when an element is referred to as being “coupled” to another element, it can describe one or more different types of coupling including, but not limited to, chemical coupling, communicative coupling, electrical coupling, electromagnetic coupling, operative coupling, optical coupling, physical coupling, thermal coupling, and/or another type of coupling. Likewise, it is to be understood that when an element is referred to as being “connected” to another element, it can describe one or more different types of connecting including, but not limited to, electrical connecting, electromagnetic connecting, operative connecting, optical connecting, physical connecting, thermal connecting, and/or another type of connecting.

The description of illustrated embodiments of the subject disclosure as provided herein, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as one skilled in the art can recognize. In this regard, while the subject matter has been described herein in connection with various embodiments and corresponding drawings, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

What is claimed is:

1. A system, comprising:
at least one processor; and
a memory that stores executable instructions that, when executed by the at least one processor, facilitate performance of operations, comprising:
receiving pathomic information, radiomic information, and transcriptomic information;
generating a multimodal model, wherein the multimodal model is generated based on combining the received pathomic information, radiomic information, and transcriptomic information;
applying patient data to the multimodal model, wherein the patient data pertains to a medical condition of a patient;
generating, based on application of the patient data to the multimodal model, a recommended treatment, wherein the recommended treatment is based on at least one node, in a sequence of nodes included in the multimodal model, having sufficient similarity to the patient data;
determining a risk score for the recommended treatment, wherein the risk score presents a measure of a successful treatment outcome for the recommended treatment regarding the patient's medical condition, wherein the risk score is determined based on applying the recommended treatment to a probability model; and
presenting the recommended treatment and risk score for review.

2. The system of claim 1, the operations further comprising:
receiving a confirmation to implement the recommended treatment; and
updating the patient data in accordance with the recommended treatment being applied to treat the patient's medical condition.

3. The system of claim 1, the operations further comprising utilizing natural language programming to facilitate interaction with the presented recommended treatment and risk score.

4. The system of claim 3, wherein the interaction is via at least one of a mouse and cursor, interactive display, or speech-based interaction.

5. The system of claim 1, wherein the multimodal model comprises at least one of a visual language model, a large language model, a Bayesian network, a convolutional neural network, graph neural network, or a knowledge graph.

6. The system of claim 1, wherein the multimodal model comprises the network of nodes and a network of edges connected to a set of potential treatments, a respective node in the network of nodes represents content pertaining to the patient's medical condition or medical knowledge regarding a medical condition, wherein the medical knowledge pertains or does not pertain to the patient's medical condition.

7. The system of claim 1, wherein the probability model comprises at least one of a visual language model, a large language model, a Bayesian network, a convolutional neural network, graph neural network, or a knowledge graph.

8. The system of claim 1, wherein the recommended treatment is a first recommended treatment and the risk score is a first risk score, wherein the operations further comprise:
generating, based on application of the patient data to the multimodal model, a second recommended treatment, wherein the second recommended treatment is based on at least one node in the sequence of nodes having sufficient similarity to the patient data;
determining a second risk score for the second recommended treatment, wherein the second risk score presents a measure of a successful treatment outcome for the second recommended treatment regarding the patient's medical condition, wherein the second risk score is determined based on applying the second recommended treatment to a probability model;
ranking the first recommended treatment and the second recommended treatment based on the first risk score and the second risk score; and
presenting the ranking of the first recommended treatment and first risk score, and the second recommended treatment and the second risk score.

9. The system of claim 1, wherein multimodal model comprises transcriptomic data pertaining to the medical condition of the patient in combination with at least one of radiology data pertaining to the medical condition of the patient, or pathology data pertaining to the medical condition of the patient.

10. The system of claim 9, wherein the transcriptomic data pertaining to the medical condition of the patient further comprises at least one of proteomic information, single-cell RNA sequencing field (scRNAseq) information, an autofluorescence image, matrix-assisted laser desorption/ionization (MALDI) information, spatial transcriptomic information, multiplexed error-robust fluorescence in situ hybridization (MERFISH), spatial gene expression, or metaboliomic information.

11. A computer-implemented method comprising:
generating, by a device comprising at least one processor, a multimodal model, wherein the multimodal model is generated based on at least one of a medical condition of a patient, historical data pertaining to the medical condition, imaging data pertaining to the medical condition, or medical knowledge pertaining to the medical condition;
determining, by the device, a potential treatment to address the medical condition of the patient, wherein the treatment is an output of the multimodal model;
determining, by the device, a probability of the potential treatment successfully addressing the medical condition of the patient, wherein the probability is determined based on application of the potential treatment to a probability model, wherein the probability model comprises a collection of treatments in conjunction with success of application of a respective treatment in the collection of treatments to a respective medical condition, wherein the probability is assigned to a risk score for the potential treatment of the patient's medical condition; and
presenting, by the device, a recommendation for treatment of the patient's medical condition, wherein the recommendation comprises the potential treatment in conjunction with the risk score.

12. The computer-implemented method of claim 11, wherein the multimodal model is one of a visual language model, a large language model, graph neural network, or a Bayesian network.

13. The computer-implemented method of claim 12, wherein the visual language model comprises transcriptomic data pertaining to the medical condition of the patient in combination with at least one of radiology data pertaining to the medical condition of the patient, or pathology data pertaining to the medical condition of the patient.

14. The computer-implemented method of claim 11, wherein the probability model is one of a visual language model, a large language model, or a Bayesian network.

15. The computer-implemented method of claim 11, wherein the multimodal model and the probability model combine to form a prediction model, the prediction model comprises at least one of a visual language model, a large language model, graph neural network, or a Bayesian network.

16. The computer-implemented method of claim 11, wherein the risk score indicates a risk stratification of at least one of the medical condition of the patient or the recommended treatment.

17. A computer program product stored on a non-transitory computer-readable medium and comprising machine-executable instructions, wherein, in response to being executed, the machine-executable instructions cause a system to perform operations, comprising:
generating a multimodal model, wherein the multimodal model is generated based on at least one of a medical condition of a patient, historical data pertaining to the medical condition, imaging data pertaining to the medical condition, or medical knowledge pertaining to the medical condition;
determining a potential treatment to address the medical condition of the patient, wherein the treatment is an output of the multimodal model;
determining a probability of the potential treatment successfully addressing the medical condition of the patient, wherein the probability is determined based on application of the potential treatment to a probability model, wherein the probability model comprises a collection of treatments in conjunction with success of application of a respective treatment in the collection of treatments to a respective medical condition, wherein the probability is assigned to a risk score for the potential treatment of the patient's medical condition; and
presenting a recommendation for treatment of the patient's medical condition, wherein the recommendation comprises the potential treatment in conjunction with the risk score.

18. The computer program product according to claim 17, wherein the multimodal model is one of a visual language model, a large language model, graph neural network, or a Bayesian network, and the probability model is one of a visual language model, a large language model, or a Bayesian network.

19. The computer program product according to claim 18, wherein the visual language model comprises transcriptomic data pertaining to the medical condition of the patient in combination with at least one of radiology data pertaining to the medical condition of the patient, or pathology data pertaining to the medical condition of the patient.

20. The computer program product according to claim 18, wherein the multimodal model and the probability model combine to form a prediction model, the prediction model comprises at least one of a visual language model, a large language model, graph neural network, or a Bayesian network.
